(19) United States
(12) Patent Application Publication
(10) Pub. No.: US 2025/0160985 A1
(43) Pub. Date: May 22, 2025

(54) MAGNETIC MICROROBOT

(71) Applicant: MULTI-SCALE MEDICAL ROBOTICS CENTER LIMITED, Hong Kong (CN)

(72) Inventors: Zhengxin YANG, Hong Kong (CN); Kai Fung CHAN, Hong Kong (CN); Li ZHANG, Hong Kong (CN)

(21) Appl. No.: 18/726,831

(22) PCT Filed: Dec. 23, 2022
(86) PCT No.: PCT/IB2022/062718

§ 371 Date: Jul. 5, 2024

Related U.S. Application Data
(60) Provisional application No. 63/297,658, filed on Jan. 7, 2022.

(51) Int. Cl. A61B 34/00; A61B 34/20; A61B 34/30; A61M 25/01; A61M 25/09

(52) U.S. Cl. CPC A61B 34/73; A61B 2034/2063; A61B 2034/301; A61B 2034/303; A61B 2034/733; A61M 25/0116; A61M 25/0127; A61M 25/09041

(57) ABSTRACT

This invention provides a microrobot. In one embodiment, said microrobot comprises: a) an attachment module (300) for connecting said microrobot to a delivery device (100); and b) a tip module (200), comprising: (i) a bullet (230), comprising an outer shell (231) and one or more first magnets, wherein said outer shell (231) has a design capable of being propelled by an external magnetic field when said one or more first magnets interacts with said external magnetic field; (ii) a holder (220) for holding said bullet comprising a release mechanism for releasing said bullet from said holder.

### FIELD OF THE INVENTION

The present invention relates to a magnetic microrobot for approaching hard-to-reach regions in tubular environments such as blood vessels.

### BACKGROUND OF THE INVENTION

Endovascular intervention is a general approach to treat vascular diseases. In the procedure, a flexible guidewire/catheter is inserted through a small incision (e.g., the femoral artery in the groin, the radial artery in the wrist) and then guided to the target lesion of the blood vessel system. However, the conventional intervention has technical challenges. The standard instrument has no active maneuverability and is operated at the remote end by push-pull and rotation. Thereby successful surgery entirely depends on the high expertise and extensive experience of the surgeon.

To date, a variety of steerable interventional devices have been designed for improved maneuverability, among which magnetic driven is one of the popular manners, and such designs are featured by miniature size, good flexibility, and low cost. Nevertheless, the successful rate through tortuous arteries and veins (e.g., curving, kinking, looping, spiral twisting) has rarely been evaluated, and these structures are reported to be challenging to manipulate and even cause instrument fracture due to the contact force between the wire and the complex blood vessel wall. On the other side, wireless miniature robots have been widely researched due to their capability of accessing complex and hard-to-reach areas, which can further perform different tasks. However, the control under fast blood flow is still challenging, and specifically, the tiny robot may get lost during swimming to the lesion or returning after treatment.

Accordingly, there is a demand for developing a device integrated with the merits of both wired and wireless miniature robots, which would be advantageous for clinical needs and helpful to the clinician for the treatment of vascular diseases. Relevant inventions remain a research gap. This invention has a compact design that consists of a commercial guidewire and an assembled tip module. The rotating locker and magnetic ejector structure enable flexible bending and controllable tip ejection, realizing two different working modalities in a single design.

### SUMMARY OF THE INVENTION

This invention provides a microrobot. In one embodiment, said microrobot comprises: a) an attachment module (300) for connecting said microrobot to a delivery device (100); and b) a tip module (200), comprising: (i) a bullet (230), comprising an outer shell (231) and one or more first magnets, wherein said outer shell (231) has a design capable of being propelled by an external magnetic field when said one or more first magnets interacts with said external magnetic field; (ii) a holder (220) for holding said bullet comprising a release mechanism for releasing said bullet from said holder.

This invention further provides a method of using the microrobot of this invention for endovascular intervention in a subject. In one embodiment, said method of using the microrobot comprises the steps of: a) Connecting said microrobot to a delivery device (100) via said attachment module (300); b) Inserting said microrobot into a vessel of said subject via an insertion point; and c) Positioning said microrobot to a suitable site, wherein forward-backward motion of said microrobot is adjusted by a motorized feeder or manually; and steering motion of said microrobot is adjusted by an external magnetic field.

This invention also provides a system for endovascular intervention in a subject. In one embodiment, said system comprises: a) the microrobot of this invention for placement at a site in said subject; b) an electromagnet array (720) for generating said external magnetic field; c) an ultrasound probe (730) for medical imaging-based feedback on position of said microrobot; and d) a parallel manipulator (710) for driving said ultrasound probe and said electromagnet array to vicinity of said site.

### BRIEF DESCRIPTION OF THE FIGURES

FIG. 1 is an illustration of actuating the guidewire attached magnetic microrobot in the vascular system. The tethered mode (upper branch) and untethered mode (lower branch) are performed under different scenarios for enhanced accessibility.

FIG. 2 is the overall structure and exploded perspective view of the guidewire attached magnetic microrobot.

FIG. 3 shows the internal states of the rotating locker and magnetic ejector under the flexible bending and tip ejection stages.

FIG. 4 shows a schematic diagram of the magnet array, computed magnetic torque and force under different stages, and distributions of magnetic field flux density with maximum attractive force and repulsive force.

FIG. 5 is kinematic analyses for tethered mode actuated by directional external magnetic fields and the untethered mode actuated by rotating external magnetic fields.

FIG. 6 shows two functionalized helical bullets: a driller-type design for blood clot removal; a porter-type design for targeted drug delivery.

FIG. 7 is a conceptual diagram of the adopted actuation system in a remote-control manner.

FIG. 8 is a block diagram showing the system structure and control process.

FIG. 9 includes the fabricated guidewire attached magnetic microrobot prototype and characterization results.

FIG. 10 contains photographs demonstrating the tethered mode in a model with multiple 3D branches, the untethered mode in a model with looping structures, and the experimental setup with respect to a life-size leg artery model.

### DETAILED DESCRIPTION OF THE INVENTION

In this invention, a guidewire attached magnetic microrobot with two working modalities is proposed for approaching hard-to-reach regions in tubular environments. The microrobot consists of a commercial guidewire and an assembled tip module. The structure of the rotating locker and magnetic ejector enables flexible bending and controllable tip ejection. The tethered mode is for navigation through the tubular network with a large diameter and high flow rate, where the forward-backward motion is controlled by the feeding device, and the steering motion is actuated by the directional external magnetic field. The untethered mode is for approaching lesions with narrowed and tortuous configuration, where the ejected helical bullet is wirelessly propelled by the rotating external magnetic field.

In this invention, a guidewire attached magnetic microrobot with two working modalities is developed, featured by good manipulability and flexibility. The invention consists of a commercial guidewire and an assembled tip module. The latter one further has three functional components, called the base frame, rotary holder, and helical bullet, which installed an array of three permanent magnets. The actuation of the microrobot relies on both well-designed mechanical structures and magnetic effects. In particular, the rotating locker enables an omnidirectional flexible bending in the tethered mode, the combination of the rotating locker and magnetic ejector fulfills controllable tip ejection for the mode switch, and the spiral configuration realizes wirelessly helical propulsion in the untethered mode.

The tethered mode is similar to a traditional intervention procedure for long-distance navigation through vascular networks but with improved manipulability due to induced magnetic navigation. This working mode has high efficiency and reliability, with the forward-backward motion controlled by adjusting the guidewire position using a feeding device, and the steering motion actuated by the directional external magnetic field. The untethered mode gets rid of the constraint of the wire, in which the released helical bullet serves as a free swimmer powered by the rotating external magnetic field, converting rotation into linear motion to go into tortuous lesions. This working mode has enhanced flexibility.

The control mode is selected according to applied environments for better performance. For example, at the beginning of the intervention, the tethered mode is adopted for navigation in vasculature with a large diameter and high blood flow for fast operating speed and avoiding loss. When it reaches the vessel with narrowed and tortuous configuration, the untethered mode is utilized, where the helical bullet is released for access to the target and retrieved after treatment.

Except for the overall structure design of the guidewire attached magnetic microrobot, relevant studies are also included: a principle of tip ejection is discussed for general design across dimensions; a kinematic model containing two modes is established for control purposes; different functionalized helical bullets are proposed for various treatment; a mobile-coil electromagnetic actuation system is adopted for human-scale validation and demonstration. The following drawings and detailed descriptions will provide a better understanding of the claimed invention.

### Definitions and Abbreviations

The following terms shall be used to describe the present invention. In the absence of a specific definition set forth herein, the terms used to describe the present invention shall be given their common meaning as understood by those of ordinary skill in the art.

As used herein, the expression directional external magnetic field refers to the applied magnetic field with arbitrary direction in all dimensions. The direction could both continuously and discretely change.

As used herein, the expression rotating external magnetic field refers to the applied magnetic field with continuously changing direction along the rotating axis. The generated magnetic field vectors in a cycle form a disc perpendicular to the rotating axis.

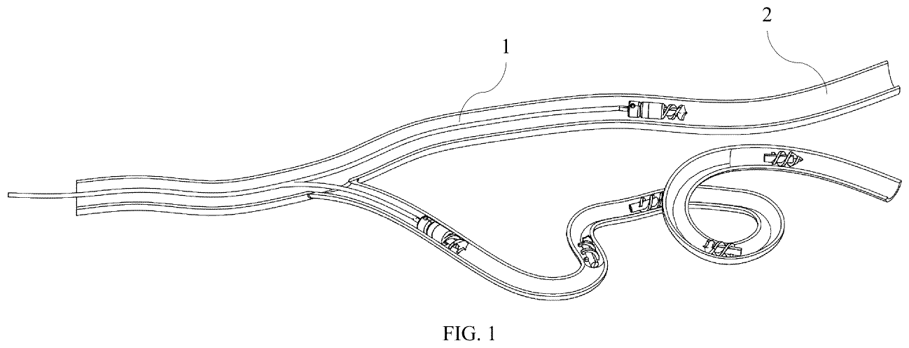

Referring to FIG. 1, the guidewire attached magnetic microrobot 1 has two working modalities which are selected according to the applied environment 2. For example, the tethered mode in the bifurcated vessel (upper branch) with fast movement speed and high reliability, and the untethered mode in the looping vessel (lower branch) for enhanced flexibility. The two-in-one design of this invention integrates the merits of both wired and wireless microrobots, which can overcome the challenge of reaching hard-to-reach regions in clinical treatment.

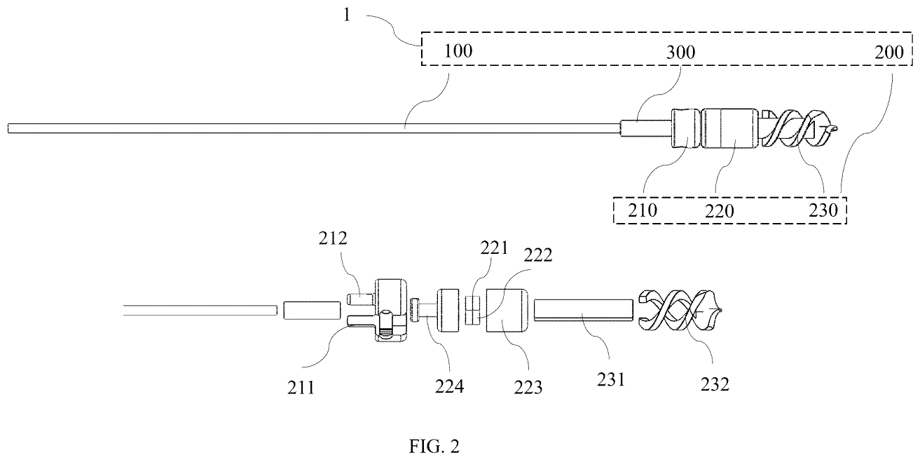

Referring to FIG. 2, the guidewire attached magnetic microrobot 1 is composed of a commercial guidewire 100 and an assembled tip module 200, with two parts connected by a segment of silicone cannula 300. The assembled tip module 200 has three functional components: base frame 210, rotary holder 220, and helical bullet 230. The base frame 210 consists of a lower stick for connecting to the guidewire 100 and an upper cavity for holding the rotary holder 220. The rotary holder 220 has a lower shaft for plugging into the base frame 210, two middle holes for installing axially magnetized cylindrical magnets with opposite magnetization directions 221, 222, and an upper bucket for accommodating the helical bullet 230. The helical bullet 230 includes an outer spiral shell 231 and an inner radially magnetized cylindrical magnet 232. The base frame 210 is separated into the left part 211 and right part 212, and the rotary holder 220 is divided into the upper part 223 and lower part 224 for assembling purposes and glued together using medical-grade adhesive.

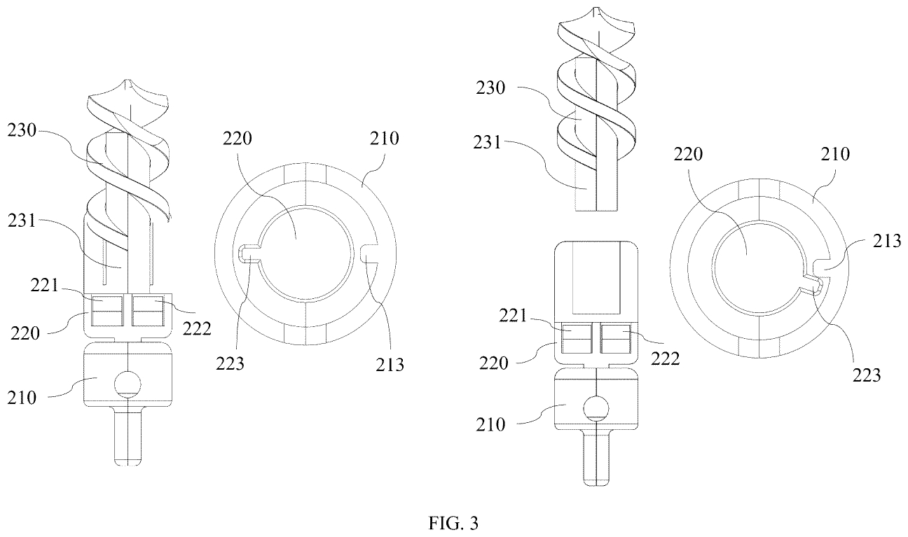

Referring to FIG. 3, the cavity of the base frame 210 has a stopper 213, and the shaft of the rotary holder has a slider 223. Such mating enables the rotary holder 220 to rotate along the axis within limits while restricting its radial deflection and axial movement. This structure is named the rotating locker. The bucket of the rotary holder 220 has an inner diameter slightly larger than the outer diameter of the helical bullet 230, which maintains the relative position between them and allows axial rotation and movement. This structure is called the magnetic ejector. Combining these two structures enables both the flexible bending for the tethered mode and the tip ejection for switching to the untethered mode.

For flexible bending, the external magnetic field is applied to ensure no blocking effect exists between the slider 223 and the stopper 213. Thereby the helical bullet 230 stably stays in the rotary holder 220 due to the attractive force between cylindrical magnets 221, 222, 231, and the combination of the rotary holder 220 and helical bullet 230 aligns with the external magnetic field for active steering. For tip ejection, the external magnetic field is first applied to make the slider 223 get stuck at the stopper 213 (either from the left side or the right side), then keeping changing the direction of the external magnetic field will only turn the helical bulled 230. When the intersection angle (δ) reaches the critical value, the helical bullet 230 will be ejected from the rotary holder 220 because of the repulsive force between cylindrical magnets 221, 222, 231. Moreover, the remainder attached to the guidewire has an approximately zero magnetic moment. Therefore it will not be affected by the external magnetic field during the following untethered mode and will wait in situ until the helical bullet 230 returns.

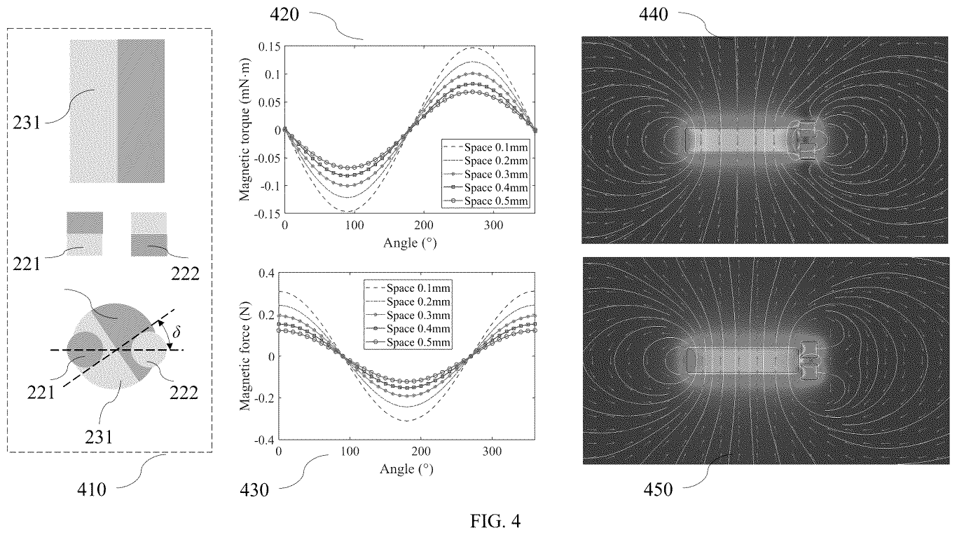

Referring to FIG. 4, tip ejection is the transition stage between two working modalities, which can be magnetically triggered. In principle, the magnetic force (Fₘ) and torque (Tₘ) applied on a magnetized object can be determined by:

$${F}_{m} = {\int }_{{V}_{m}} ( m \cdot ∇ ) B ( x , y , z ) {dV}_{m} \\ {T}_{m} = {\int }_{{V}_{m}} m \times B ( x , y , z ) {dV}_{m}$$

where Vₘ is the volume of the magnetized object; m is the magnetization vector; B(x,y,z) is the magnetic field at (x,y,z). The abstract diagram of mounted cylindrical magnets is shown 410, where dark gray and light gray represent N-pole and S-pole, respectively. Since the lower two cylindrical magnets 221, 222 are fixed in the rotary holder 220, only the magnetic force/torque that the lower magnet set 221, 222 exerts on the upper magnet 231 is considered when it comes to the inter-magnet effect. Dipole approximation provides convenient analytical properties and is an excellent fit to deal with permanent magnet problems at large distances. However, the installed cylindrical magnets 221, 222, 231 are close to each other, leading to the above approximation being inaccurate.

In this disclosure, finite element analyses (FEA) simulation is conducted (COMSOL Multiphysics 5.3a, COMSOL Inc.). Examples of computed magnetic torques and forces on magnet 231 under various δ are shown 420, 430. Results show that the interaction force changes between attractive and repulsive during rotation. Also, the interaction moment reaches the maximum at δ equals 90° and 270°. Furthermore, the magnetic field distribution maps when δ equals 0° for attractive and 180° for repulsive are shown 440, 450. In addition, external magnetic fields are applied to turn the helical bullet 230, and the tip can be ejected by the repulsive force between cylindrical magnets 221, 222, 231.

For the prototype guidewire attached magnetic microrobot, the dimensions of cylindrical magnets are first selected according to existing product models. Then other parameters are decided by parametric sweep to balance maximum attractive force and effective external magnetic field turning effect for simultaneous stable angle steering and magnetically triggered ejection. This procedure can be used to design a series of customized guidewires across dimensions.

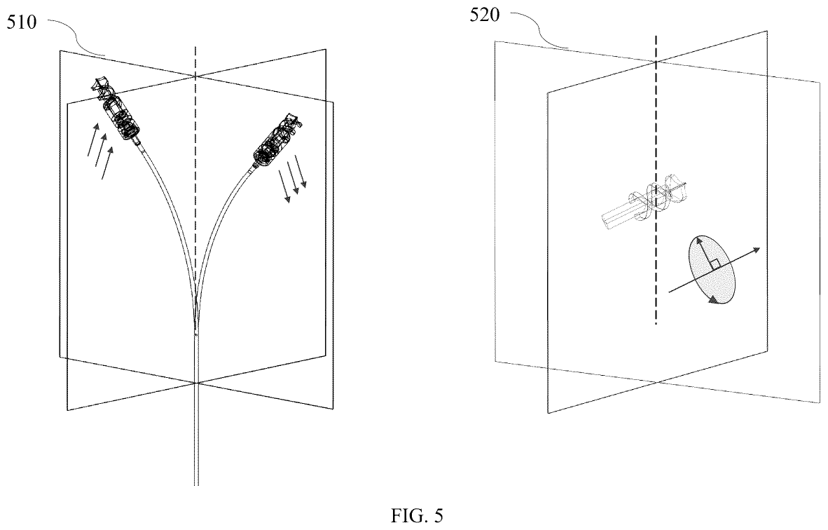

Referring to FIG. 5, the proposed guidewire attached magnetic microrobot has two working modalities. In the tethered mode, the rotary holder 220 and helical bullet 230 can be viewed as an entity. As shown 510, directional external magnetic fields (Ba) are imposed:

$${B}_{d} = {{A}_{m} [ sin β cos α sin β s in α cos β ]}^{T}$$

where Aₘ, α, and β are the strength, yaw angle, and pitch angle of the directional external magnetic field, respectively. The applied field aligns the magnetization of the tip in the bending plane, and the induced magnetic torque (T_{d}) equals:

$$\vert {T}_{d} \vert = \vert M \vert \vert {B}_{d} \vert sin ( β - θ - π / 2 )$$

where M is the magnetic moment of the assembled tip; θ is the bending angle of the guidewire attached magnetic microrobot. The restoring torque (Tₑ) leading the guidewire attached magnetic microrobot to the original state can be calculated as:

$$\vert {T}_{e} \vert = {EI}_{a} θ / {L}_{c}$$

where E is the elastic modulus; Iₐ is the area moment of inertia; L_{c} is the length of the deformation segment. The equilibrium state achieves when |T_{d}|=|Tₑ|, which is used to predict deformation.

In the untethered mode, rotating external magnetic fields (Bᵣ) expressed as:

$${B}_{r} = {A}_{m} ( cos ( 2 π ft ) {u}_{r} + ( sin ( 2 π ft ) ) {n}_{r} \times {u}_{r} )$$

are applied that result in a synchronous revolution of the helical bullet 230, as illustrated 520, where f is the rotating frequency; nᵣ is the unit vector of the rotating axis; uᵣ is the corresponding normal vector in the propulsion plane:

$${n}_{r} = {[ sin β c os α sin β sin α cos β ]}^{T} \\ \\ \\ {u}_{r} = {[ - cos β cos α - cos β sin α sin β ]}^{T}$$

Mimicking corkscrew-type microorganisms, the helical bullet 230 can convert rotation into linear motion and propels along tubular environments.

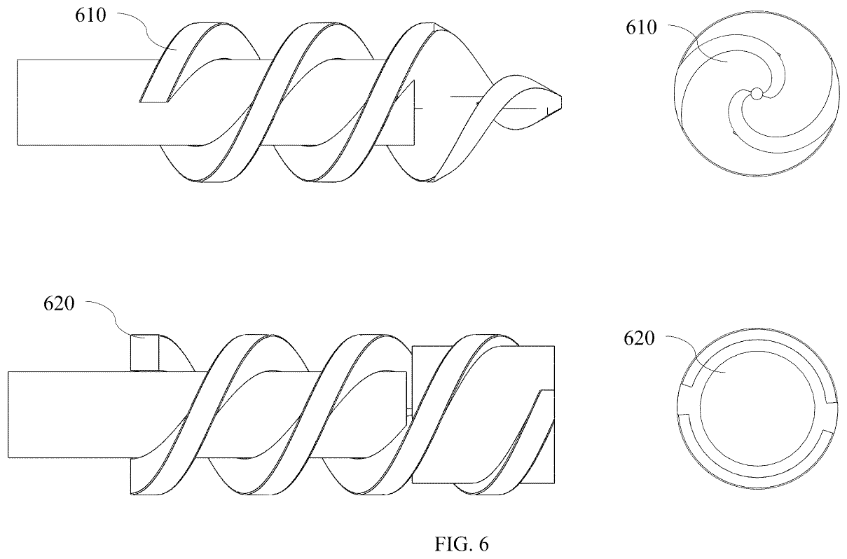

Referring to FIG. 6, the mentioned helical bullet can be modified to integrate with various functions for different therapies. One example is the driller-type design 610, which is used to treat thrombus and recover blood flow. It has an outer shell with a tapered head. When it reaches the clogged region, high-speed drilling can be performed by applying high-frequency rotating external magnetic fields, and the blood clot can be removed by mechanical rubbing. Another example is the porter-type design 620, which can be used for targeted drug delivery. It has an outer shell with a loading cavity to carry drugs. Combined with material technique, drug delivery can be realized at the desired lesion under environmental stimulation.

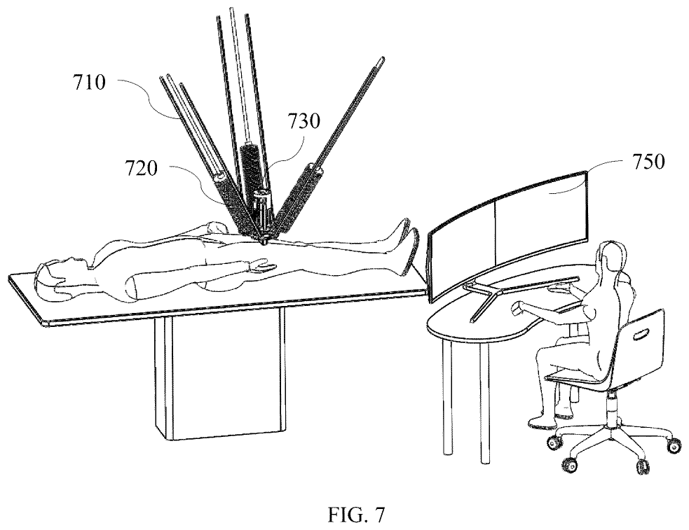

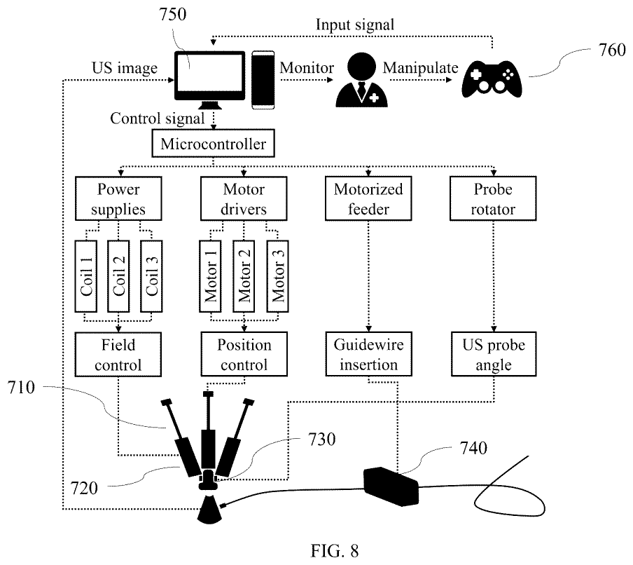

Referring to FIG. 7 and FIG. 8, this invention also proposes a remote control method. Currently, we use the so-called DeltaMag System for actuation, which has an array of three mobile electromagnetic coils, and other systems are also applicable. A conceptual diagram including the patient, the surgeon, and the actuating system is given. A homemade system comprising parallel manipulator 710 and electromagnet array 720 is adopted for large-workspace magnetic field generation. Furthermore, a 2D US probe 730 is mounted on the mobile endplate for medical imaging-based feedback, and a motorized feeder 740 is designed for the forward-backward motion of the guidewire. During actuation, the parallel manipulator 710 has 3 degrees of freedom (DOFs) to drive the US probe 730 and the electromagnet array to the vicinity of the device, the US probe 730 has 1 DOF for adjusting the imaging view angle, and the electromagnet array 720 has 3 DOFs to create arbitrary magnetic fields. All modules are coordinately controlled through a user interface 750 with commands inputted by a joystick 760.

This invention provides a guidewire attached magnetic microrobot. In one embodiment, said guidewire attached magnetic microrobot comprises: a commercial guidewire without active steering ability; an assembled tip module with varying magnetic responses under different external magnetic fields.

In one embodiment, the assembled tip module comprises a body that includes different functional components, such as base frame, rotary holder, and helical bullet.

In one embodiment, the assembled tip module comprises a base frame comprising a lower stick for connecting to the guidewire and an upper cavity for holding the rotary holder.

In one embodiment, the assembled tip module comprises a rotary holder having a lower shaft for plugging into the base frame, two middle holes for installing magnets, and an upper bucket for accommodating the helical bullet.

In one embodiment, the assembled tip module comprises a helical bullet comprising an outer spiral shell and an inner magnet.

In one embodiment, the assembled tip module comprises a helical bullet that can be modified and integrated with different functionalization, such as blood clot removal, targeted drug delivery, biopsy, and embolization.

In one embodiment, the assembled tip module utilizes a propelling strategy of the helical bullet can be modified into other mechanisms, such as flexible-ora type, vibrating type, and climbing type.

In one embodiment, the commercial guidewire and the assembled tip module can be connected through various methods, such as silicone cannula and tiny spring.

In one embodiment, the assembled tip module comprises a rotating locker that enables omnidirectional flexible bending, and the magnetic ejector fulfills controllable tip ejection for the mode switch.

In one embodiment, the rotating locker comprises a mechanism utilizing the stopper of the base frame and the slider of the rotary holder shaft, enabling the rotary holder to rotate along the axis within limits while restricting its radial deflection and axial movement.

In one embodiment, the magnetic ejector comprises a mechanism that requires the rotary holder bucket to have an inner diameter slightly larger than the outer diameter of the helical bullet, maintaining the relative position between the helical bullet and the rotary holder and allowing axial rotation and movement.

In one embodiment, the magnetic ejector uses the attractive and repulsive force between magnets for holding and releasing the helical bullet relates to the intersection angle, and the intersection angle is controlled by external magnetic fields.

In one embodiment, the magnetic ejector has comprises a magnetic array where the dimensions and relative positions of the magnet array can be redesigned, matching with overall dimension requirement and actuating magnetic field.

In one embodiment, the magnetic ejector comprises a magnet array that is not limited to cylindrical magnets but also other ferromagnetic substances with various shapes and fabrication methods.

In one embodiment, the guidewire attached magnetic microrobot has two working modalities and a magnetically triggered switch: the tethered mode; the tip ejection stage; and the untethered mode.

In one embodiment, the tethered mode controls the forward-backward motion by insertion and retrieval of the guidewire, and the steering motion is actuated by the directional external magnetic field.

In one embodiment, the tethered mode performs the forward-backward motion manually or robotically.

In one embodiment, the tip ejection stage comprises firstly locking the rotating locker, and the intersection angle is then changed.

In one embodiment, the untethered mode comprises wirelessly propelling the ejected helical bullet by the rotating external magnetic field.

In one embodiment, in the untethered mode, the remainder attached to the guidewire after ejection has an approximately zero magnetic moment.

In one embodiment, in the untethered mode, the helical bullet can swim back and be retrieved by the guidewire.

In one embodiment, the guidewire attached magnetic microrobot is for approaching hard-to-reach regions in tubular environments, such as the blood vessel system, digestive tract system, and urinary system.

In one embodiment, the guidewire attached magnetic microrobot is remotely controlled using an operating system comprising: a magnetic field generating module, wherein the equipment with electromagnetic coils or permanent magnets generates directional and rotating external magnetic fields; a guidewire insertion module, wherein the device performs robotic guidewire insertion and retrieval with both speed and distance control; an imaging feedback module, wherein the method comprises standard medical imaging methods, such as X-ray fluoroscopy, computed tomography (CT), and ultrasonography; and a user control module, wherein a controller is for user command input, such as a joystick and a 5D mouse, and a programmed user interface for monitoring.

This invention provides a microrobot. In one embodiment said microrobot comprises: a) an attachment module (300) for connecting said microrobot to a delivery device (100); and b) a tip module (200), comprising: (i) a bullet (230), comprising an outer shell (231) and one or more first magnets, wherein said outer shell (231) has a design capable of being propelled by an external magnetic field when said one or more first magnets interacts with said external magnetic field; (ii) a holder (220) for holding said bullet comprising a release mechanism for releasing said bullet from said holder.

In one embodiment, said delivery device (100) is a guidewire or catheter.

In one embodiment, said bullet (230) has a functionalized design selected from the group consisting of a driller-type design and porter-type design.

In one embodiment, said bullet (230) is propelled by said external magnetic field using a propelling strategy selected from the group consisting of spiral type, flexible-ora type, vibrating type, and climbing type.

In one embodiment, said outer shell (231) is a spiral or helical shell capable of turning rotation into linear motion.

In one embodiment, said attachment module (300) is a cannula or spring.

In one embodiment, said one or more first magnets (232) comprises a radially magnetized cylindrical magnet.

In one embodiment, said release mechanism comprises: a) one or more second magnets (221, 222) in said holder (220); and b) a configuration for controlling relative movements between said one or more first magnets (232) and said one or more second magnets (221, 222) so that magnetic repulsive force can be generated to release said bullet (230) from said holder (220).

In one embodiment, said configuration comprises: a) a cylindrical bucket in said holder (220) for receiving said bullet (230); b) a cylindrical portion in said bullet (230) for insertion into said cylindrical bucket, wherein said bullet (230) can rotate within said holder (220) when a suitable external magnetic field is applied; and c) a blocking mechanism that can be activated to prevent rotation of said one or more second magnets (221, 222) under said suitable external magnetic field. In another embodiment, said blocking mechanism comprises: a first component comprising a slider (223); and a second component comprising a stopper (213), wherein said one or more second magnets (221, 222) are attached to said first component or second component, said first component and said second component is configured to rotate about a same axis and no relative motion between said first component and said second component can occur when said slider (223) meets said stopper (213). In a further embodiment, said one or more second magnets (221, 222) comprise two axially magnetized cylindrical magnets with opposite magnetization directions.

This invention further provides a method for endovascular intervention in a subject using the microrobot of this invention. In one embodiment, said method comprises the steps of: a) Connecting said microrobot to a delivery device (100) via said attachment module (300); b) Inserting said microrobot into a vessel of said subject via an insertion point; and c) Positioning said microrobot to a suitable site, wherein forward-backward motion of said microrobot is adjusted by a motorized feeder or manually; and steering motion of said microrobot is adjusted by an external magnetic field.

In one embodiment, said method further comprises the step of activating said release mechanism to release said bullet (230) from said holder (220). In another embodiment, said method further comprises controlling movement of said bullet (230) using an external magnetic field. In a further embodiment, said method further comprises the step of controlling said bullet (230) to reattach to said holder (220).

This invention also provides a system for endovascular intervention in a subject. In one embodiment, said system comprises: a) the microrobot of this invention for placement at a site in said subject; b) an electromagnet array (720) for generating said external magnetic field; c) an ultrasound probe (730) for medical imaging-based feedback on position of said microrobot; and d) a parallel manipulator (710) for driving said ultrasound probe and said electromagnet array to vicinity of said site.

In one embodiment, said system further comprises a delivery device (100) attached to said microrobot. In another embodiment, said system further comprises a motorized feeder (740) for adjusting forward-backward motion of said delivery device (100). In a further embodiment, said bullet (230) is propelled by said external magnetic field using a propelling strategy selected from the group consisting of flexible-ora type, vibrating type, and climbing type.

In one embodiment, said release mechanism comprises: a) one or more second magnets (221, 222) in said holder (220); and b) a configuration for controlling relative movements between said one or more first magnets (232) and said one or more second magnets (221, 222) so that magnetic repulsive force can be generated to release said bullet (230) from said holder (220).

The invention will be better understood by reference to the Experimental Details which follow, but those skilled in the art will readily appreciate that the specific experiments described are only for illustrative purposes and are not meant to limit the invention as described herein, which is defined by the claims that follow thereafter.

Throughout this application, various references or publications are cited. Disclosures of these references or publications in their entireties are hereby incorporated by reference into this application in order to more fully describe the state of the art to which this invention pertains. It is to be noted that the transitional term “comprising”, which is synonymous with “including”, “containing” or “characterized by”, is inclusive or open-ended and does not exclude additional, un-recited elements or method steps.

### Example 1

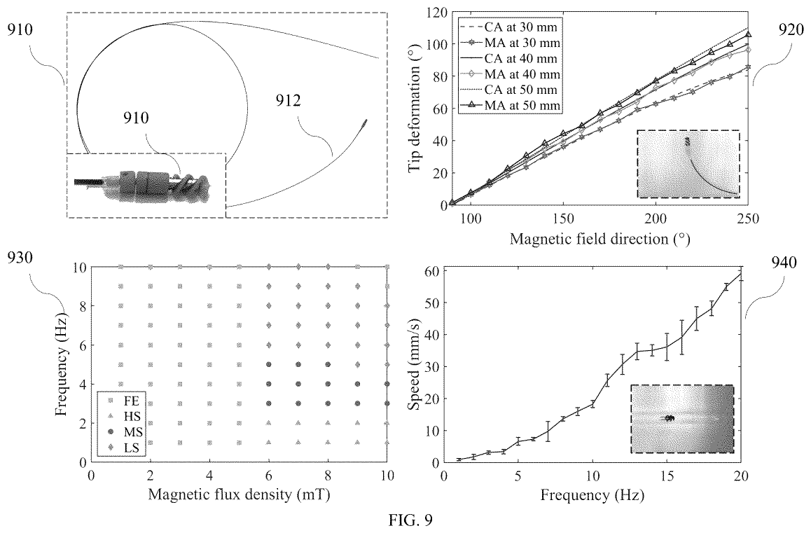

Referring to FIG. 9, a guidewire attached magnetic microrobot prototype 910 was fabricated, and corresponding characterizations were conducted. The components of the current prototype were processed by 3D printing, and other fabrication methods will be utilized in the future. The assembled tip 911 had an outer diameter of 2.8 mm and a length of 12.5 mm; the attached commercial guidewire 912 had an outer diameter of 0.025 in and a length of 1500 mm.

Plot 920 shows the comparison between computed and measured bending angles of the guidewire attached magnetic microrobot under various directional external magnetic fields with a magnitude of 9 mT. Different lengths of deformation segments were included, including 30 mm, 40 mm, and 50 mm. The maximum bending error was 4.5° and the average error was 1.9°. These showed the effectiveness of the proposed kinematic model.

Plot 930 demonstrates the performance of tip ejection. For convenient operation, the tip ejection was triggered by the rotating external magnetic field with the rotating axis along the centerline, after which a command was given to stop the field. Results showed that the low-frequency medium-strength rotating external magnetic field was preferred. When the magnitude of the external magnetic field was small, for example, from 1 mT to 5 mT, the external magnetic torque was not strong enough to change the existing internal attraction state, so the tip ejection failed. When the frequency of the rotating external magnetic field was high, for example, more than 6 Hz, the helical bullet 230 could be captured by the rotary holder 220 during the ejection process due to the rapidly alternative intersection angle, which caused the reduced success rate. These validated the mechanism of tip ejection.

Plot 940 displays the motion speed of the ejected helical bullet 230 under the rotating external magnetic field. The frequency varied from 1 Hz to 20 Hz with a 1 Hz increment, and the test was repeated three times. The forward velocity increased with the rotating frequency. These verified the wireless propulsion.

### Example 2

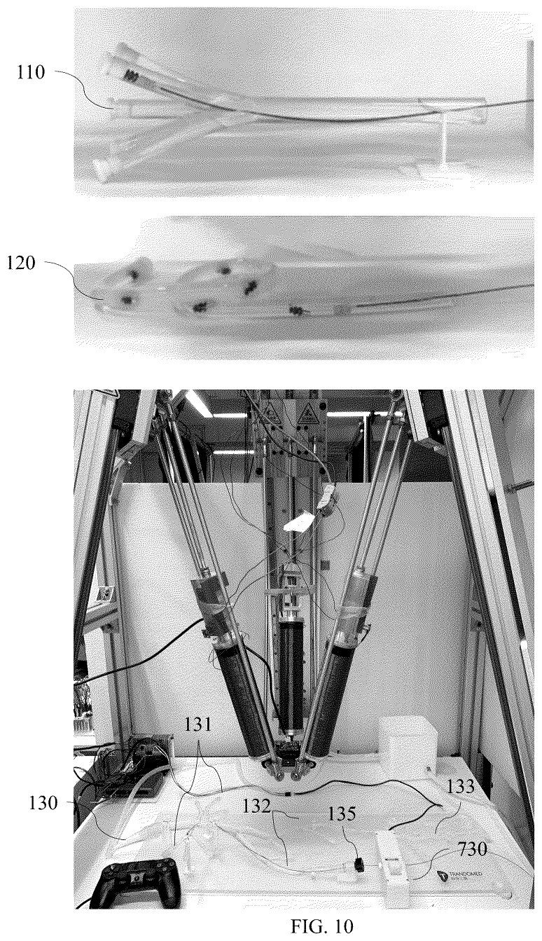

Referring to FIG. 10, the in-vitro test results were provided. Two tubular models were designed by modeling software and fabricated by 3D printing to validate different working modalities.

The first tubular model 110 had five branches distributed uniformly in the 3D space. The guidewire attached magnetic microrobot could be easily inserted into an arbitrary channel under the guidance of a directional external magnetic field. The results showed good maneuverability in the tethered mode.

The second tubular model 120 had a looping structure. Tip ejection was triggered when the guidewire attached magnetic microrobot reached the target position. After this, rotating external magnetic fields were applied so that the helical bullet 230 was wirelessly propelled through the tortuous structure. If the whole procedure was performed in the tethered mode, keeping inserting the guidewire attached magnetic microrobot with directional external magnetic fields steering the orientation, the guidewire attached magnetic microrobot could get stuck in the circular region. The results proved the enhanced flexibility in the untethered mode.

### Example 3

A life-size leg artery phantom 130 was applied, which was fabricated based on real CT data for physiological fidelity. This phantom was clinically employed for training the surgeons on the interventional procedure. The overall dimension of the model was 715 mm×239 mm×100 mm (length×width×height), with inner diameters ranging from 3 mm to 20 mm. Different paths were designed, including the renal artery 131, iliac artery 132, and femoral artery 133. The guidewire attached magnetic microrobot prototype 910 was inserted through insertion point 135.

The tethered mode was first utilized, with forward-backward motion controlled by the motorized feeder 730 and turning operation guided by the directional external magnetic field. The guidewire attached magnetic microrobot first went through the left and right renal arteries successively. Afterward, it returned to the bifurcated region and oriented to the iliac artery 132. Tip ejection was prepared to be triggered when it came to the relatively narrowed vessel. Next, the rotating external magnetic field was applied to eject the helical bullet 230. Finally, the untethered mode was adopted for reaching deep lesions 133. The demonstration on the phantom validated the effectiveness of the invention over a long-distance operation.

### Example 4

The performance of the invention in the environment with flow rate is tested. The disturbances induced by the flow should not have a great effect on the tethered mode. While for the tip ejection and the untethered mode, the influence of the flow can be mitigated by combination with functional catheters, such as a balloon catheter, to stabilize the guidewire and temporarily interrupt the blood flow.

Various guidewire attached magnetic microrobot prototypes are designed and fabricated across dimensions to fit different working scenarios. Ex-vivo and in-vivo experiments using medical imaging methods for monitoring are conducted to validate the invention.

1-20. (canceled)

21. A microrobot, comprising:
a. an attachment module (300) for connecting said microrobot to a delivery device (100); and
b. a tip module (200), comprising:
i. a bullet (230), comprising an outer shell (231) and one or more first magnets, wherein said outer shell (231) has a design capable of being propelled by an external magnetic field when said one or more first magnets interacts with said external magnetic field;
ii. a holder (220) for holding said bullet comprising a release mechanism for releasing said bullet from said holder;
wherein said release mechanism comprises:
one or more second magnets (221, 222) in said holder (220); and
a configuration for controlling relative movements between said one or more first magnets (232) and said one or more second magnets (221, 222) so that magnetic repulsive force can be generated between said one or more first magnets (232) and said one or more second magnets (221, 222) to release said bullet (230) from said holder (220).

22. The microrobot of claim 21, wherein said delivery device (100) is a guidewire or catheter.

23. The microrobot of claim 21, wherein said bullet (230) has a functionalized design selected from the group consisting of a driller-type design and porter-type design.

24. The microrobot of claim 21, wherein said bullet (230) is propelled by said external magnetic field using a propelling strategy selected from the group consisting of spiral type, flexible-ora type, vibrating type, and climbing type.

25. The microrobot of claim 21, wherein said outer shell (231) is a spiral or helical shell capable of turning rotation into linear motion.

26. The microrobot of claim 21, wherein said attachment module (300) is a cannula or spring.

27. The microrobot of claim 21, wherein said one or more first magnets (232) comprises a radially magnetized cylindrical magnet.

28. The microrobot of claim 21, wherein said configuration comprises:
a. a cylindrical bucket in said holder (220) for receiving said bullet (230);
b. a cylindrical portion in said bullet (230) for insertion into said cylindrical bucket, wherein said bullet (230) can rotate within said holder (220) when a suitable external magnetic field is applied; and
c. a blocking mechanism that can be activated to prevent rotation of said one or more second magnets (221, 222) under said suitable external magnetic field.

29. The microrobot of claim 28, wherein said blocking mechanism comprises:
a. a first component comprising a slider (223); and
b. a second component comprising a stopper (213);
wherein said one or more second magnets (221, 222) are attached to said first component or second component, said first component and said second component is configured to rotate about a same axis and no relative motion between said first component and said second component can occur when said slider (223) meets said stopper (213).

30. The microrobot of claim 21, wherein said one or more second magnets (221, 222) comprise two axially magnetized cylindrical magnets with opposite magnetization directions.

31. A method of using the microrobot of claim 21 for endovascular intervention in a subject, comprising the steps of:
a. Connecting said microrobot to a delivery device (100) via said attachment module (300);
b. Inserting said microrobot into a vessel of said subject via an insertion point; and
c. Positioning said microrobot to a suitable site, wherein forward-backward motion of said microrobot is adjusted by a motorized feeder or manually; and steering motion of said microrobot is adjusted by an external magnetic field.

32. The method of claim 31, further comprising the step of activating said release mechanism to release said bullet (230) from said holder (220).

33. The method of claim 32, further comprises controlling movement of said bullet (230) using an external magnetic field.

34. The method of claim 32, further comprises the step of controlling said bullet (230) to reattach to said holder (220).

35. A system for endovascular intervention in a subject, comprising:
a. the microrobot of claim 21 for placement at a site in said subject;
b. an electromagnet array (720) for generating said external magnetic field;
c. an ultrasound probe (730) for medical imaging-based feedback on position of said microrobot; and
d. a parallel manipulator (710) for driving said ultrasound probe and said electromagnet array to vicinity of said site.

36. The system of claim 35, further comprises a delivery device (100) attached to said microrobot.

37. The system of claim 36, further comprises a motorized feeder (740) for adjusting forward-backward motion of said delivery device (100).

38. The system of claim 35, wherein said bullet (230) is propelled by said external magnetic field using a propelling strategy selected from the group consisting of flexible-ora type, vibrating type, and climbing type.
